(19) United States
(12) Patent Application Publication
(10) Pub. No.: US 2003/0213925 A1
(43) Pub. Date: Nov. 20, 2003

(54) Activity detector circuit

(76) Inventors: Fouad Kiamilev, Hockessin, DE (US); Theodore J. (Ted) Wyman, Mont Vernon, NH (US)

Correspondence Address: MORGAN & FINNEGAN, L.L.P., 345 Park Avenue, New York, NY, 10154-0053, US

(21) Appl. No.: 10/389,840

(22) Filed: Mar. 17, 2003

Related U.S. Application Data
(60) Provisional application No. 60/365,603, filed on Mar. 18, 2002.

(51) Int. Cl. G21K 4/00; G03C 5/16; A61B 6/00; G01N 23/04

(52) U.S. Cl. 250/581

(57) ABSTRACT

An activity detection system for use with an optoelectronic circuit has a pair of single ended transimpedance amplifiers, a filter, a differential amplifier; and a comparator.

### CROSS REFERENCE TO RELATED APPLICATIONS

This application claims priority under 35 USC 119(e)(1) of U.S. Provisional Patent Application Serial No. 60/365,603 filed Mar. 18, 2002.

### FIELD OF THE INVENTION

The present invention relates generally to detection circuits, and relates more particularly to activity detector circuits for use with optical devices.

### BACKGROUND

Activity detection systems can be used to monitor the voltage or current levels of a particular system. However, often times these activity detection systems are simple comparators that compare the voltage or current level against a predetermined threshold level that cannot be easily changed.

In the field of optical electronics, activity detectors are often used with optical receiving circuits so the activity of the optical signal can be detected. FIG. 1 is a typical optical receiving circuit of the prior art that inputs the optical signal into a single ended amplifier 110. The amplified signal is averaged using filter 120 and both the signal and the filtered average is fed into differential amplifier 130. A differential amplifier instead of a singled ended amplifier is used because a differential amplifier reduces noise in the signal.

Another implementation of the receiving circuit is shown in FIG. 2. A constant input 204 is generated from a dummy photodetector and is used along with the signal 202. Both the signal 202 and the constant input 204 are amplified by single ended amplifiers 206 and 208 respectively, and the outputs from the single ended amplifiers are filtered by the filter 210. The filter averages the signal, and removes some of the noise present in the signals. The noise-reduced outputs from the filters are then fed into the differential amplifier 212. This implementation uses a common filter for both inputs and a single capacitor is used for both differential signals so that the chip area necessary for this circuit is reduced.

### SUMMARY OF THE INVENTION

We have devised an invention that allows creation of an activity detection system having a variable threshold and that can be coupled with receiving circuits of optical devices or other differential amplifier circuits.

The advantages and features described herein are a few of the many advantages and features available from representative embodiments and are presented only to assist in understanding the invention. It should be understood that they are not to be considered limitations on the invention as defined by the claims, or limitations on equivalents to the claims. For instance, some of these advantages are mutually contradictory, in that they cannot be simultaneously present in a single embodiment. Similarly, some advantages are applicable to one aspect of the invention, and inapplicable to others. Thus, this summary of features and advantages should not be considered dispositive in determining equivalence. Additional features and advantages of the invention will become apparent in the following description, from the drawings, and from the claims.

### BRIEF DESCRIPTION OF THE DRAWINGS

FIG. 1 is a block diagram of an optical receiver amplifier circuit of the prior art;

FIG. 2 is a block diagram of another more fully differential optical receiver amplifier circuit of the prior art;

FIG. 3 is an example of a graph for the operation of an activity detection system according to the present invention;

FIG. 4 is an example of a graph for an output of the differential amplifier circuit of FIG. 2 when used in connection with the invention;

FIG. 5 is a block diagram of one embodiment of the activity detection system according to the present invention; and

FIG. 6 is a block diagram of another embodiment of the activity detection system according to the present invention.

### DETAILED DESCRIPTION

We have created an activity detection system that has a user controllable variably settable threshold.

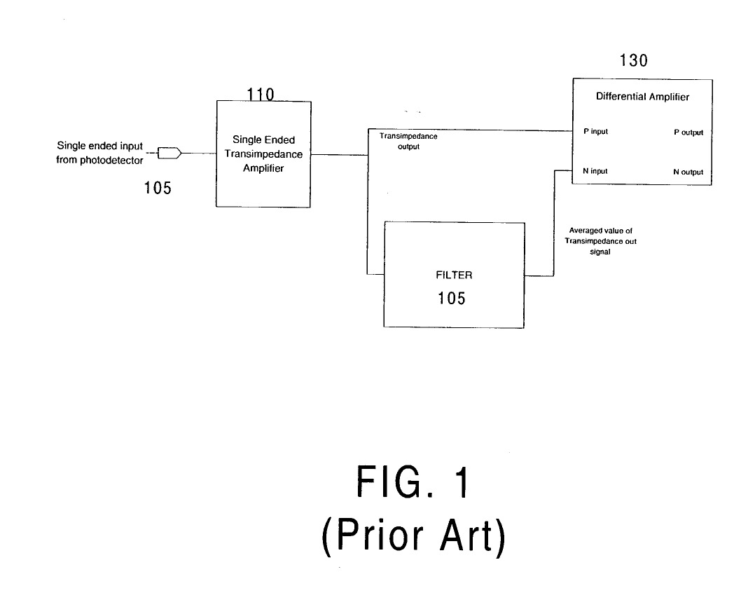

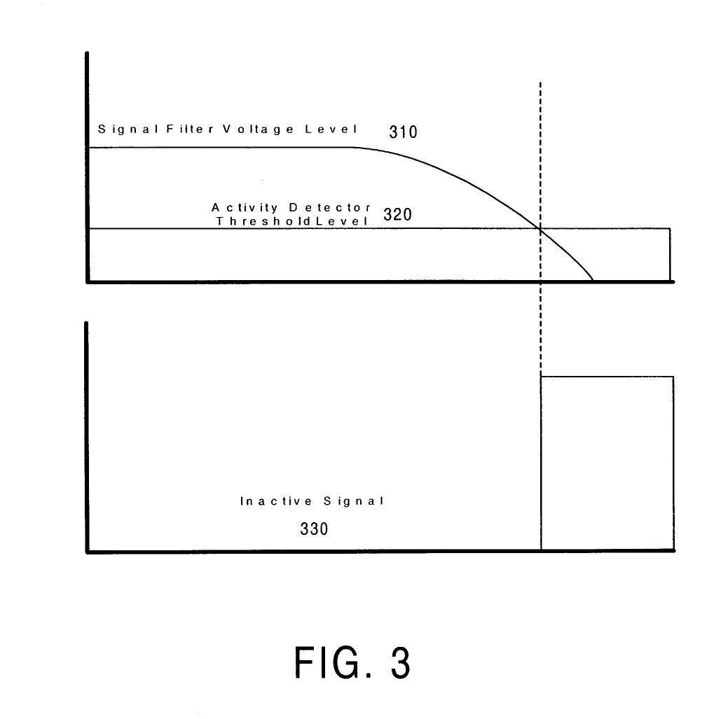

FIG. 3 is a graph of an operation of an activity detection system according to the present invention used with the optical receiver circuit having a singled ended pre-amplifier of FIG. 1. The filtered average value 310 from filter 120 is compared to a threshold value 320. The threshold value is controlled by a user and may be generated, for example, by a digital to analog converter (DAC). When the signal is no longer present at the optical input 105, because the optical transmitter has ceased transmitting or because the optical fiber is no longer connected to the optical input, the filtered signal value 310 which was indicating the average value of the input signal will begin to fall. A threshold level is set such that when the input signal is no longer present the average value falls below it and thus, the comparator output will indicate inactivity. A threshold value is applied to one end of a comparator and the filtered value is applied to the other end so that when the signal is present the average will be above the threshold and thereby the comparator will indicate that activity is present.

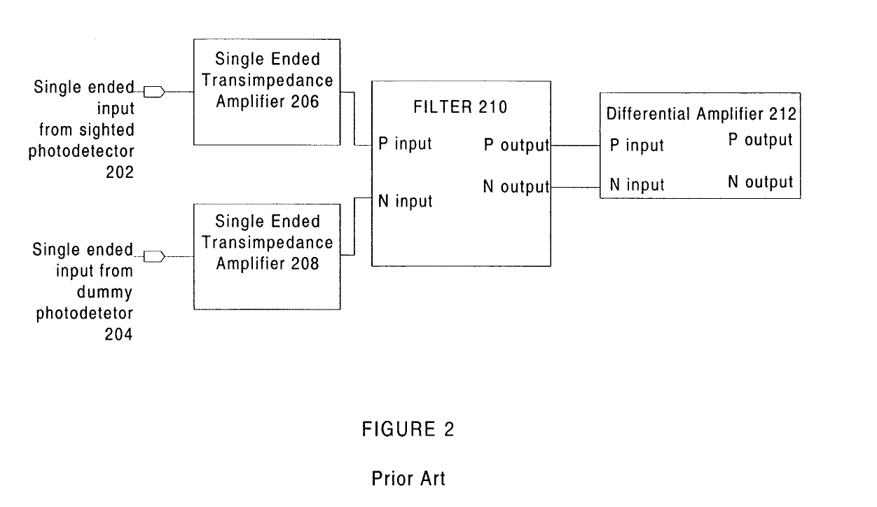

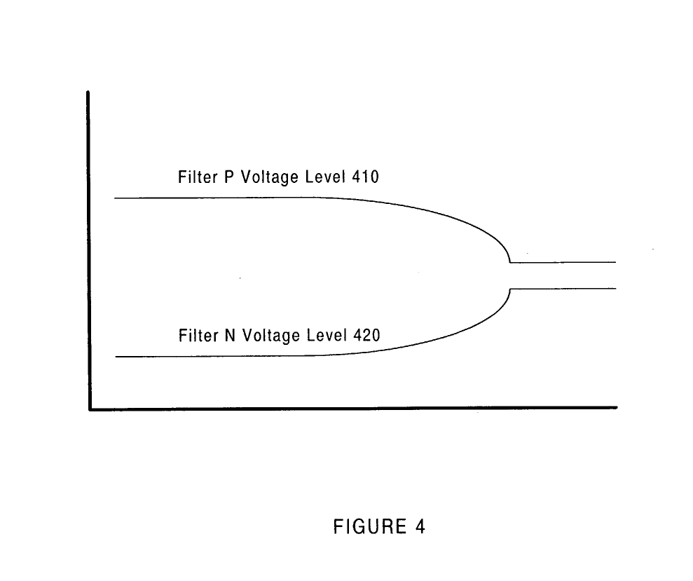

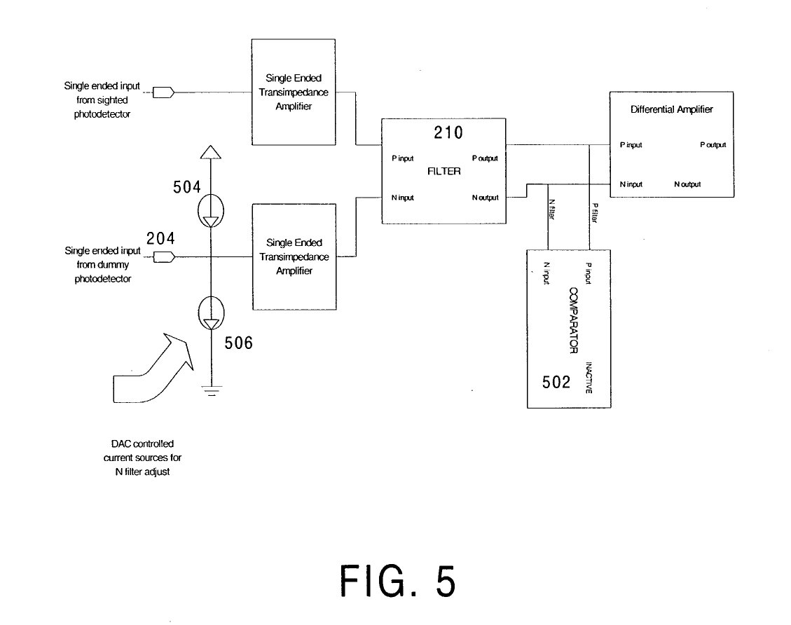

Another embodiment of the activity detection system of the present invention can be used with the differential circuit of FIG. 2 in order to take full advantage of the double input filter 210. FIG. 5 is a block diagram of such an embodiment. The embodiment of FIG. 5, takes advantage of the output of the filter 210 of FIG. 2 that shown in FIG. 4. As activity stops in the input signal 202 and dummy signal 204, the filter P voltage level 410 and filter N voltage level 420 will decay and approach each other until they are at approximately the same voltage. The embodiment of the activity detection system of the present invention shown in FIG. 5 takes advantage of this, A comparator 502 is connected to the outputs of the filter 210. A current injector circuit made with current source 504 and current sink 506 is connected to the dummy signal 204. By varying the current at the dummy input, filter N voltage level 420 can be made to rise and fall, and the comparator 502 can determine if the input signal is inactive. For example, if the filter N voltage level is higher than the filter P voltage level, then the comparator can determine that the input signal is inactive. The current injector circuit is also controlled by a user through the use of a DAC, or the current injector circuit may be incorporated into a DAC. The current injector circuit may or may not need to have the ability to both sink or source current into the dummy input.

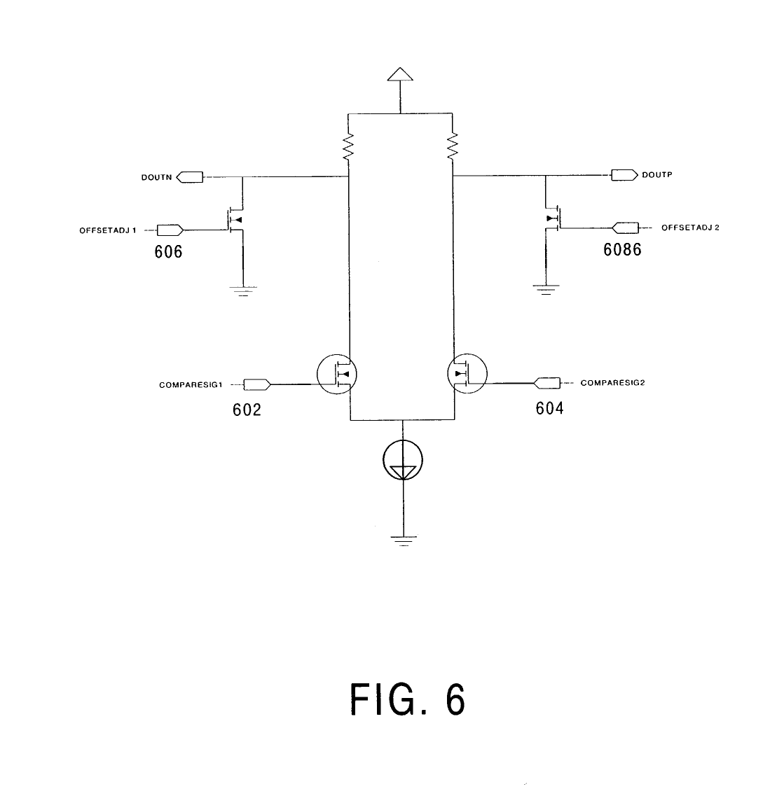

FIG. 6 is a block diagram of another embodiment of the activity detection system of the present invention. This embodiment of the present invention is a comparator that can also be used with the differential circuit of FIG. 2 and is connected so that the inputs COMPARESIG1 602 and COMPARESIG2 604 are connected to the P and N output of the filter 210. The activity detection function is accomplished by introducing an offset in the comparator by using the inputs labeled OFFSETADJ1 606 and OFFSETADJ2 608. For example, when the voltage at the gate of OFFSETADJ1 606 is increased, the voltage at the N side of the comparator will decrease, effectively creating an offset in the comparator. Then the filter N voltage level 420 can be compared with filter P voltage level 410 to determine if an inactivity event has occurred. The embodiment shown in FIG. 6 creates an offset in the comparator itself, because the level the filter N voltage 420 must reach before the comparator will switch is increased by the amount of voltage shift created by OFFSETADJ1 606. Furthermore, the embodiment shown in FIG. 6 only uses current sources. As a result, the DAC that can be used to control OFFSETADJ1 606 and OFFSETADJ2 608 can be made smaller than the one used in the embodiment shown in FIG. 5, where both a current source and a current sink may be necessary.

It should be understood that the above description is only representative of illustrative embodiments. For the convenience of the reader, the above description has focused on a representative sample of all possible embodiments, a sample that teaches the principles of the invention. The description has not attempted to exhaustively enumerate all possible variations. That alternate embodiments may not have been presented for a specific portion of the invention, or that further undescribed alternate embodiments may be available for a portion, is not to be considered a disclaimer of those alternate embodiments. One of ordinary skill will appreciate that many of those undescribed embodiments incorporate the same principles of the invention and others are equivalent.

What is claimed is:

1. An activity detection system for use with an optoelectronic circuit, comprising:
a pair of single ended transimpedance amplifiers, each having an input and an output,
a) a first of the inputs being configured to receive a first signal from a first photodetector that receives optical input;
b) a second of the inputs being configured to receive a second signal from a dummy photodetector;
a filter, having filter inputs and filter outputs, coupled to the outputs of the single ended transimpedance amplifiers;
a differential amplifier coupled to the filter outputs; and
a comparator, coupled to the filter outputs, and configured to determine, by comparison, when the first signal is below a variable threshold level established by the dummy signal and thereby to indicate that the first signal is an inactive signal.

2. The activity detection system of claim 1 wherein the compartator further comprises:
a first offset adjustment; and
a second offset adjustment.

3. The activity detection system of claim 1 further comprising a digital to analog converter coupled to the first and second offset adjustments.
